(19) United States
(12) Patent Application Publication
(10) Pub. No.: US 2009/0157069 A1
(43) Pub. Date: Jun. 18, 2009

(54) Systems and methods for thermal treatment of body tissue

(76) Inventors: Curtis Tom, San Mateo, CA (US); Raj Subramaniam, Fremont, CA (US); Mathieu Herbette, Eclepens (CH); Chrysbe Madayag, San Mateo, CA (US)

Correspondence Address: PATENT OFFICE OF DR. CHUNG S. PARK, P. O. BOX 62312, SUNNYVALE, CA, 94088-2312, US

(21) Appl. No.: 12/315,360

(22) Filed: Dec. 2, 2008

Related U.S. Application Data
(60) Provisional application No. 60/992,771, filed on Dec. 6, 2007.
(60) Provisional application No. 60/992,764, filed on Dec. 6, 2007.
(60) Provisional application No. 60/992,756, filed on Dec. 6, 2007.
(60) Provisional application No. 60/992,761, filed on Dec. 6, 2007.
(60) Provisional application No. 60/992,768, filed on Dec. 6, 2007.

(51) Int. Cl. A61B 18/18; A61B 6/00; A61F 7/12

(52) U.S. Cl. 606/33; 600/431; 604/113

(57) ABSTRACT

Apparatus and methods for treating body tissue by use of thermal treatment material. The thermal treatment material to be injected into target tissue of a body includes: a carrier substrate; a plurality of first particles operative to generate thermal energy in response to an alternating electromagnetic field applied external to the body; and a plurality of second particles, each of the second particles having a core and a coating surrounding the core. The coating is dissolved at a preset temperature by the thermal energy so that the visibility of the core in an external imaging system is affected as the coating is dissolved to expose the core. The variation of the visibility can be used as an indicator to determine if the material has reached the preset temperature.

### CROSS REFERENCE TO RELATED APPLICATIONS

This application claims the benefits of U.S. Provisional Application No. 60/992,771, entitled “Inductively heated materials with therapeutic properties” by Herbette et al., filed on Dec. 6, 2007, U.S. Provisional Application No. 60/992,764, entitled “A method of applying thermal energy into neural tissue” by Herbette et al., filed on Dec. 6, 2007, U.S. Provisional Application No. 60/992,756, entitled “Method for treating intraductal breast cancer by hyperthermia” by Herbette et al., filed on Dec. 6, 2007, U.S. Provisional Application No. 60/992,761, entitled “Method for treating human body cavity by hyperthermia” by Herbette et al., filed on Dec. 6, 2007, and U.S. Provisional Application No. 60/992,768, entitled “Method and apparatus for applying thermal energy to arterial and venous lumens and structures” by Herbette et al., filed on Dec. 6, 2007.

### BACKGROUND

The present disclosure relates to medical methods and apparatus for treating various types of biological cells and tissue by inducing localized hyperthermia or thermal ablation.

Humans and/or animals can suffer from various types of tissue-related illnesses, such as varicose veins, breast cancer, and tumors. One of the approaches in treating these diseases is with thermotherapy. Thermotherapy subjects tissue(s) to temperatures that result in structural modification, damage or destruction of cells that comprise the tissue. One method of thermotherapy, hyperthermia, employs miniscule particles that are capable of converting electromagnetic energy into thermal energy. These particles are delivered to the target tissue and destroy the malignant cells with thermal energy when the particles are immersed in an alternating magnetic field. Thermotherapy may be used in thermal ablation by raising cell or tissue temperature to a point where physical cell destruction occurs. Hereinafter, the term tissue collectively refers to a portion of a body to be treated.

Existing methods of inducing hyperthermia and thermal ablation employ radio frequency (RF) currents, microwave energy, photonic energy, ultrasonic energy and the cauterization to the targeted tissue/cells. In all of these modalities, energy delivery and thermo-regulation are critical parameters since excessive energy absorption may result in unintended and/or collateral damage to adjacent tissue or structures and undesired char formation. Typical shortcomings in some of these technologies include large and non-conforming electrodes or applicators (electrodes and applicators are the devices that delivery energy from a source to the tissue) and complex temperature sensing and controlling schemes.

The bilateral tubal sterilization (TS) is another well known thermotherapy technique. In developed countries, permanent tubal occlusion is most commonly performed using laparoscopy techniques (utilizing a transabdominal approach) where the fallopian tubes are physically occluded using a ring, a clip or electrocauterization. An estimated 700,000 bilateral TS are performed annually in the US and 11 million US women 15-44 years of age rely on TS for contraception. Tubal sterilization has also been shown to be associated with decreased risk of ovarian cancer.

Despite its worldwide use and high efficacy, TS using the transabdominal approach is associated with substantial trauma and discomfort which, in a majority of cases, involves the inconvenience and expense of a hospital stay and carries the risk of complications such as bleeding, infection, bowel perforation and reaction to general anesthesia. A few transcervical tubal occlusion devices have been developed and are steadily gaining acceptance as a viable alternative to transabdominal sterilization techniques.

Available tubal blocking systems depend upon mechanical occlusive techniques, chemically or thermally induced tissue damage and combinations of these techniques. Chemical agents induce tissue damage, which leads to formation of scar tissue to seal the opening of the fallopian tubes. The major drawback to this method is the need for repeated applications. Thermal blocking systems use either heat or cryogenic methods to damage tissue and also induce the formation of scar tissue to seal the opening of the fallopian tubes.

### SUMMARY OF THE DISCLOSURE

In one embodiment, a material to be injected into target tissue of a body includes: a carrier substrate; a plurality of first particles operative to generate thermal energy in response to an alternating electromagnetic field applied external to the body; and a plurality of second particles, each of the second particles having a core and a coating surrounding the core. The coating is dissolved at a preset temperature by the thermal energy so that the visibility of the core in an external imaging system is affected as the coating is dissolved to expose the core. The variation of the visibility can be used as an indicator to determine if the material has reached the preset temperature.

### BRIEF DESCRIPTION OF THE DRAWINGS

FIGS. 1A-1D show various types of material for thermal treatment of body tissue in accordance with one embodiment of the present invention;

FIG. 2A shows a schematic side view of a catheter inserted into a varicose vein in accordance with another embodiment of the present invention;

FIG. 2B shows a schematic side view of the catheter in FIG. 2A during treatment of the varicose vein;

FIG. 3A shows a schematic side view of a catheter inserted into a varicose vein in accordance with another embodiment of the present invention;

FIG. 3B shows a schematic side view of the catheter in FIG. 3A during treatment of the varicose vein;

FIG. 4A shows a schematic side view of a catheter in accordance with another embodiment of the present invention;

FIG. 4B shows a schematic cross sectional view of the catheter in FIG. 4A positioned in a vein;

FIG. 4C shows a schematic cross sectional view of the catheter in FIG. 4A during treatment of the vein;

FIG. 5A shows a schematic cross sectional view of a catheter in accordance with another embodiment of the present invention;

FIG. 5B shows a schematic side view of the catheter in FIG. 5A applied to thermally treat a uterus;

FIG. 5C shows a flow chart illustrating steps to treat the uterus in FIG. 5B in accordance with another embodiment of the present invention;

FIG. 5D shows a flow chart illustrating steps to inject thermal treatment material into the uterine cavity of FIG. 5B in accordance with another embodiment of the present invention;

FIG. 6A shows a schematic cross sectional view of a catheter in accordance with another embodiment of the present invention;

FIG. 6B shows a schematic side view of the catheter in FIG. 6A applied to thermally treat a uterus;

FIG. 7A shows a schematic cross sectional view of a human breast with a catheter inserted thereto for thermal treatment in accordance with another embodiment of the present invention;

FIG. 7B shows a flow chart illustrating steps to treat the human breast in FIG. 7A in accordance with another embodiment of the present invention;

FIG. 7C shows a flow chart illustrating steps to inject thermal treatment material into the human breast in FIG. 7A in accordance with another embodiment of the present invention;

FIG. 8A shows a flow chart illustrating steps to treat neural tissue in accordance with another embodiment of the present invention;

FIG. 8B shows a flow chart illustrating steps to treat neural tissue in accordance with another embodiment of the present invention;

FIG. 8C shows a flow chart illustrating steps to treat neural tissue in accordance with another embodiment of the present invention;

FIG. 9A shows a schematic cross sectional view of a uterus with a catheter inserted thereinto for thermal treatment of fallopian tubes in accordance with another embodiment of the present invention;

FIG. 9B shows a schematic cross sectional view of a uterus and fallopian tubes containing ferrite material for thermal treatment thereof in accordance with another embodiment of the present invention;

FIG. 9C shows a schematic cross sectional view of a uterus and fallopian tubes containing ferrite materials and plugs for thermal treatment thereof in accordance with another embodiment of the present invention;

FIG. 9D shows a schematic cross sectional view of a uterus and fallopian tubes containing plug units and a plug for thermal treatment thereof in accordance with another embodiment of the present invention;

FIG. 9E shows a schematic side view of a plug for thermal treatment of a fallopian tube in accordance with another embodiment of the present invention; and

FIG. 9F shows a schematic end view of the plug in FIG. 9E.

### DETAILED DESCRIPTION OF THE PREFERRED EMBODIMENTS

The following detailed description is of the best currently contemplated modes of carrying out the invention. The description is not to be taken in a limiting sense, but is made merely for the purpose of illustrating the general principles of the invention because the scope of the invention is best defined by the appended claims.

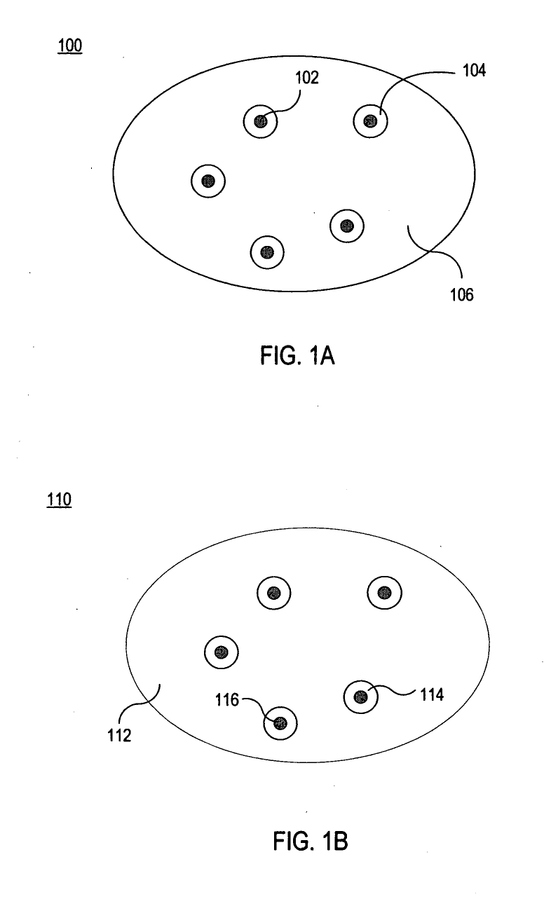

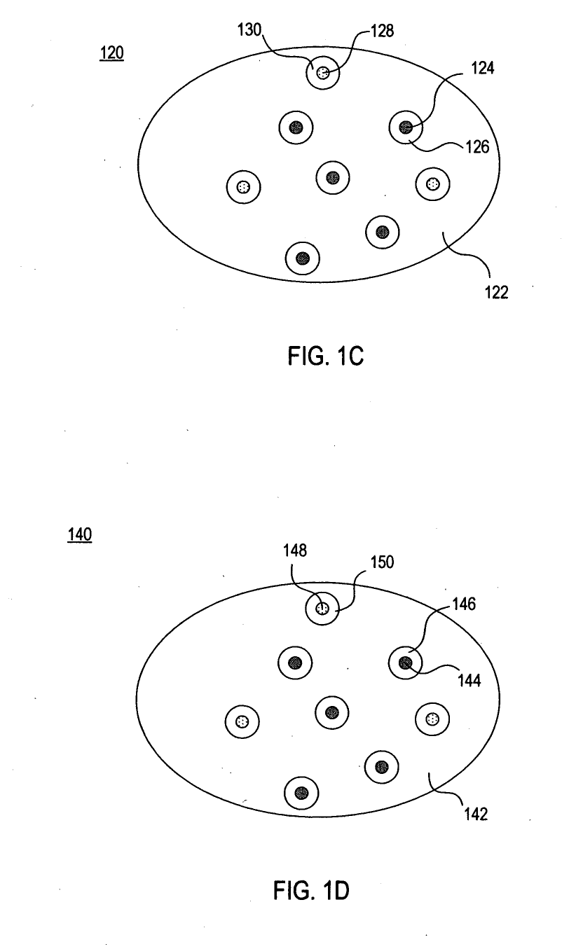

FIG. 1A shows a material 100 for thermal treatment of body tissue in accordance with one embodiment of the present invention. As depicted, the thermal treatment material (or, shortly, material) 100 includes a carrier substrate 106 and particles (or cores) 102 with particle coating 104. The particles 102 may be formed of biocompatible or bio-absorbable material, and generate heat energy when excited externally by alternating electromagnet (EM) field. The apparatus disclosed in U.S. patent application Ser. No. 11/823,379, entitled “Systems and methods for inductive heat treatment of body tissue,” can be used to generate the external alternating electromagnetic field. The material 100 may be embedded within a tool, such as a catheter or a probe disclosed in U.S. patent application Ser. No. 11/801,453, entitled “Systems and Methods for Treating Body Tissue” and Ser. No. 11/823,380, entitled “Systems and methods for delivering particles into patient body,” and injected into the human body via the catheter. U.S. patent application Ser. Nos. 11/823,379, 11/823,380 and 11/801,453 are incorporate herein by reference in their entirety.

The carrier substrate 100 may be biocompatible, bio-absorbable, and can be formulated as a liquid, gel, solid, or some permutation thereof, depending on the type of application. The carrier substrate 100 provides additional properties, such as anti-clumping, anesthetic, promotion of flow and coverage, and promotes visualization and other therapeutic agents. The carrier substrate 100, for instance, can be formulated with a polymer such as polyglycolic acid to produce a solid, bio-absorbable implantable. The particles 102, suspending in the carrier substrate 106, may be composed of ferrimagnetic, ferromagnetic, or super-paramagnetic materials. The particles 102 have an average size from 1 nm to 100 μm so as to induce a high specific absorption rate (SAR) in the tissue. The particle coating 104 may be anionic or cationic and include organic or inorganic compounds at a thickness of 1 nm-100 μm, and provide biocompatibility and prevent particle agglomeration. The particle coating 104 may have an additional coating of surfactant.

The material 100 is inherently thermally self-regulating to prevent the temperature of the material from exceeding the designed-in upper temperature limit. For instance, the particles 102 may be formed of a metal alloy with a preset Curie temperature so that the temperature of the material does not go beyond the Curie temperature during operation. The Curie temperature is lower than the threshold temperature to damage healthy cells and higher than the threshold temperature to destroy malignant cells.

FIG. 1B shows a material 110 for thermal treatment of body tissue in accordance with another embodiment of the present invention. As depicted, the material 110 includes a carrier substrate 112 and particles (or cores) 116 with coating 114. The material 110 is similar to the material 100 in FIG. 1A, with the difference that the coating 114 contains substances to serve as therapeutic agents when dissolved within the human body. The therapeutic agent may include, for instance, one or more of Doxorubicin family, Paclitaxel, and Tomaxifen and aid in patient treatment, recovery or comfort (i.e. wound healing, pain management).

FIG. 1C shows a material 120 for thermal treatment of body tissue in accordance with another embodiment of the present invention. As depicted, the material 120 includes a carrier substrate 122, particles (or cores) 124 with coating 126, and contrast agents (or cores) 128 with contrast agent coating 130. The carrier substrate 122, the particles 124, and the particle coating 126 are similar to their counterparts in FIG. 1A, and, thus, detailed description of these components are not repeated for brevity.

The contrast agents 128 serve as a contrast agent and/or to improve visualization under ultrasound, fluoroscopy, MRI and other suitable imaging techniques. For instance, the contrast agents block the passage of X-rays to result in bright areas in a conventional x-ray image, promote the reflection of ultrasonic energy waves back to the source to result in an increase of ultrasonic signal intensity of the area containing the substrate, or alter the relaxation times of the excited spins in the MRI technique thereby to increase or decrease the signal intensity of the area containing the substrate. By use of the visualization/imaging technique with the contrast agent 128, a medical practitioner can easily determine the location of particles within the human body. The contrast agents 128 may be formed of Gadolinium based material, Methylxanthines, or N-acetylcysteine, for instance.

The contrast agents 128 may be coated with contrast agent coating 130 such that at a specific temperature, the coating 130 will release the contrast agent 128 to indicate the target temperature has been achieved as an aid to the operator. Alternatively, the coating 130 may release a secondary substance to inhibit the contrast agent functionality. The sizes of the particles 124 and the particle coating 126 may be in the same ranges as those of the contrast agents 128 and the contrast agent coating 130, respectively.

FIG. 1D shows a material 140 for thermal treatment of body tissue in accordance with another embodiment of the present invention. As depicted, the material 140 includes a carrier substrate 142, particles (or cores) 144 with coating 146, and therapeutic agents (or cores) 148 with therapeutic agent coating 150. The carrier substrate 142, the particles 144, and the particle coating 146 are similar to their counterparts in FIG. 1A, and, thus, detailed description of these components are not repeated for brevity.

The therapeutic agents 148 contain chemical compounds that can aid in patient recovery and comfort (i.e. wound healing, pain management). The therapeutic agents 148 and the therapeutic agent coating 150 operate as a drug delivery agent. The therapeutic agent coating 150 will release or activate the therapeutic agents 148 to optimize the therapeutic effect. The therapeutic agents themselves may be heat activated or enhanced by temperatures above 37° C., for instance. The sizes of the particles 148 and the particle coating 146 may be in the same ranges as those of the therapeutic agents 148 and the therapeutic coating 150, respectively.

It should be apparent to those of ordinary skill that the thermal treatment materials depicted in FIGS. 1A-1D may contain various combinations of particles. For instance, the material 140 may also include the contrast agents 128 with the contrast agent coating 130. Also, the particle coating 146 may contain other therapeutic materials.

The material for the carrier substrate, such as 106, 112, 122, and 142, can be selected so that the optical properties of the carrier substrate may vary with temperature. This feature can be used as a temperature indicator when using ultrasound, fluoroscopy, MRI and other imaging techniques. Also, the material for the carrier substrate may be selected so that the viscosity of the carrier substrate can be increased to the point of becoming a viscoelastic solid when a static external magnetic field is applied thereto. For instance, the static magnetic field may be applied to a target location so that the particles, such as 102, formed of metal and contained in the carrier substrate, can be disposed within the location.

The carrier substrates 106, 112, 122, and 142 may be in the form of a fluid with a viscosity of 0.3×10⁻³-50 PaS. The material for the carrier substrate may be selected such that the interaction between the carrier substrate and the particles suspended in the carrier substrate can hold the carrier substrate within the target location, too, i.e., the thermal treatment materials, such as 100, 110, 120, and 140, can have a property of a viscoelastic solid. By applying the magnetic field, the thermal treatment materials can be maintained at the target location for treatment.

The carrier substrates 106, 112, 122, and 142 may incorporate additional agent(s) to improve penetration into a fine cavity, where the additional agent can be an organic compound (e.g., surfactant) which will reduce the surface tension on the tissue. Alternatively, the carrier substrates may include additional agent(s) to improve adhesion to the tissue.

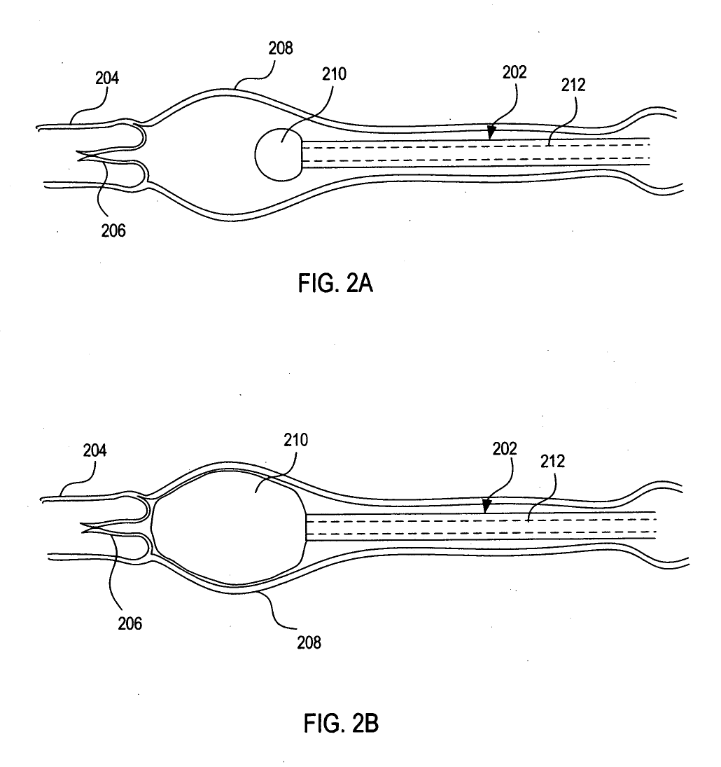

A venous system consists of a network of lumens and numerous venous valves that serve to prevent retrograde blood flow to the heart. These valves permit the flow of blood in one direction only (away from the heart). Varicose veins are the result of bicuspid valve(s) failure and/or dilatation of superficial veins in the venous system. Unlike existing treatment modalities, such as ligation of the damaged lumen (surgical, chemically, or with RF energy), surgical valve repair, grafting vein sections from other areas, and elevation of the legs and using elastic support hose, the thermal treatment materials 100, 110, 120, and 140 are use to treat various types of tissue, such as vein wall and venous valves, in a minimally invasive manner. FIG. 2A shows a schematic side view of a catheter 202 inserted into a varicose vein 204 in accordance with another embodiment of the present invention. FIG. 2B shows a schematic side view of the catheter 202 during thermal treatment of the varicose vein 204.

As depicted in FIG. 2A, the catheter 202 includes a ductal lumen 212 and a balloon 210 located at the tip of the catheter and connected to the distal end of the ductal lumen. A physician may insert the catheter 202 into the vein 204 so that the balloon 210 is positioned near the weakened wall portion 208. Then, by use of an injection mechanism (not shown in FIGS. 2A-2B) connected to the proximal end the ductal lumen 212, the thermal treatment material (such as 100, 110, 120, and 140) is injected into the balloon 210 via the ductal lumen until the balloon 210, preferably formed of polymer, is expanded to a proper size, as shown in FIG. 2B. Detailed description of the injection mechanism can be found in the previously referenced U.S. patent application Ser. No. 11/823,380.

Upon inflating the balloon 210, an external alternating electromagnetic field is applied to the thermal treatment material in the balloon 210 so that the particles (such as 102, 116, 124, and 144) contained in the thermal treatment material convert the electromagnetic field energy into thermal energy. The generated thermal energy may be used to shrink the weakened wall portion 208 in order to restore the functionality of the vein. As the venous wall 208 shrinks due to the application of heat energy, the pressure on the balloon 210 will cause the balloon to slowly deflate to maintain a constant and optimal pressure.

The balloon 210 can conform to any structures within the venous system and therefore provide optimal thermal transfer to the target tissue, such as the weakened wall portion 208 and valve leaflets 206. Also, the thermal treatment material is capable of delivering precise thermal energy, minimizing the formation of undesired heat lesions, char, or blood coagulation. More detailed information of the balloon 210 and catheter 202 can be found in the previously referenced U.S. patent application Ser. No. 11/801,453.

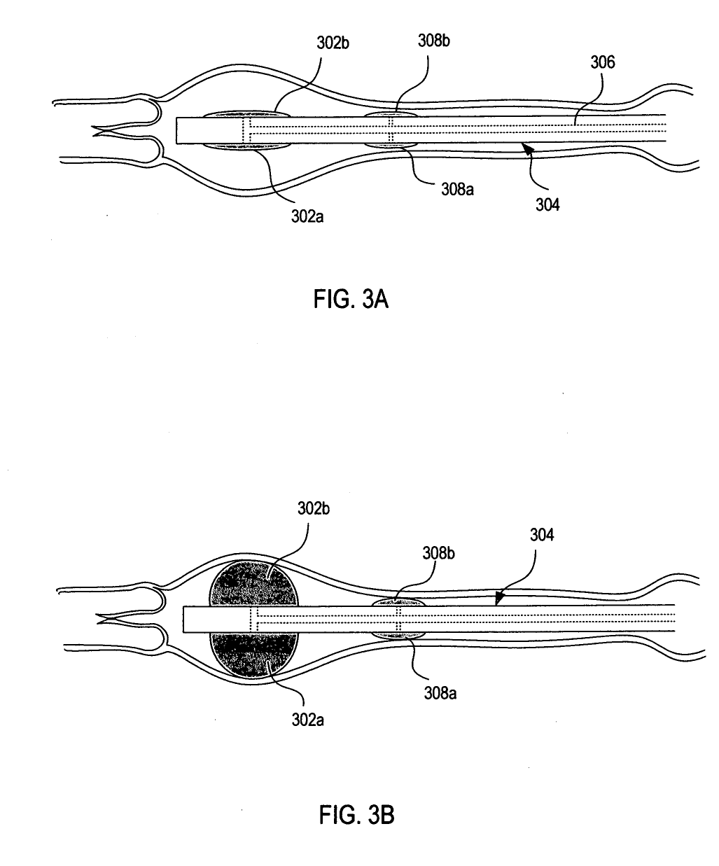

FIG. 3A shows a schematic side view of a catheter 304 inserted into a varicose vein in accordance with another embodiment of the present invention.

FIG. 3B shows a schematic side view of the catheter 304 during thermal treatment of the varicose vein. As depicted, the catheter 304 includes a first set of balloons 302a, 302b and a second set of balloons 308a, 308b. The first set of balloons 302a, 302b are connected to a ductal lumen 306 formed in the catheter 304. The second set of balloons 308a, 308b are connected to another ductal lumen (not shown in FIGS. 3A-3B) formed in the catheter 304 in the similar manner as the first set of balloons are connected to the ductal lumen 306.

Upon inflating the two sets of balloons, an external alternating electromagnetic field is applied to the thermal treatment material in the first set of balloons 302a, 302b so that the particles (such as 102, 116, 124, and 144) contained in the thermal treatment material convert the electromagnetic field energy into thermal energy. The second set of balloons 308a, 308b are inflated to temporarily block the blood flow, to thereby minimize conductive and convective heat loss during the treatment.

In FIGS. 3A and 3B, only two balloons 302a, 302b are used to generate thermal energy. However, it should be apparent to those of ordinary skill in the art that any other suitable number and shape of balloons can be used without deviating from the spirit of the present invention. For instance, a ring shaped balloon may be used in place of the first set of balloons.

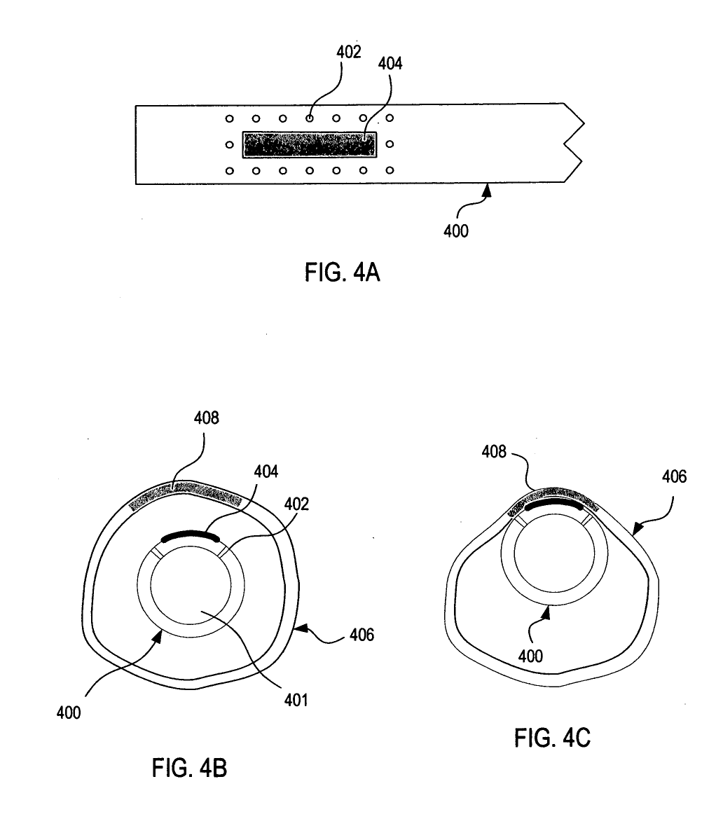

FIG. 4A shows a schematic side view of a catheter 400 in accordance with another embodiment of the present invention. FIG. 4B shows a schematic cross sectional view of the catheter 400 positioned in a vein 406. FIG. 4C shows a schematic cross sectional view of the catheter 400 during thermal treatment of a target portion 408 of the vein 406.

As depicted, the catheter 400 includes suction holes 402 formed in the wall thereof and a heat generator 404 formed along the wall thereof. The suction holes 402 are connected to the ductal lumen 401 in the catheter 400. The catheter 400 may be connected to a vacuum system (not shown in FIGS. 4A-4C) to permit heat generator 404 to adhere firmly against the target portion 408 for optimal heat transfer during thermal treatment, as shown in FIG. 4C.

The heat generator 404, formed of ferrimagnetic, ferromagnetic, or super-paramagnetic materials, converts external alternating electromagnetic energy into thermal energy. The heat generator 404 can be formulated so that it is thermally self regulating and able to control the energy delivered to the target portion 408. The dimension, shape, and pattern of the heat generator 404 may be determined based on the shape and extent of the target tissue, such as the target vein wall portion 408 and the valve leaflets 206 (FIG. 2A). Exemplary catheters with heat generators are disclosed in the previously referenced U.S. patent application Ser. No. 11/823,380 and No. 11/801,453.

Conventional imaging technologies (ultrasound, fluoroscopy, etc.) may be used to position the catheter/heat generator into position within the vein 406. Both the catheter 400 and the heat generator 404 may be formed of materials to aid in imaging and navigation through the vein 406.

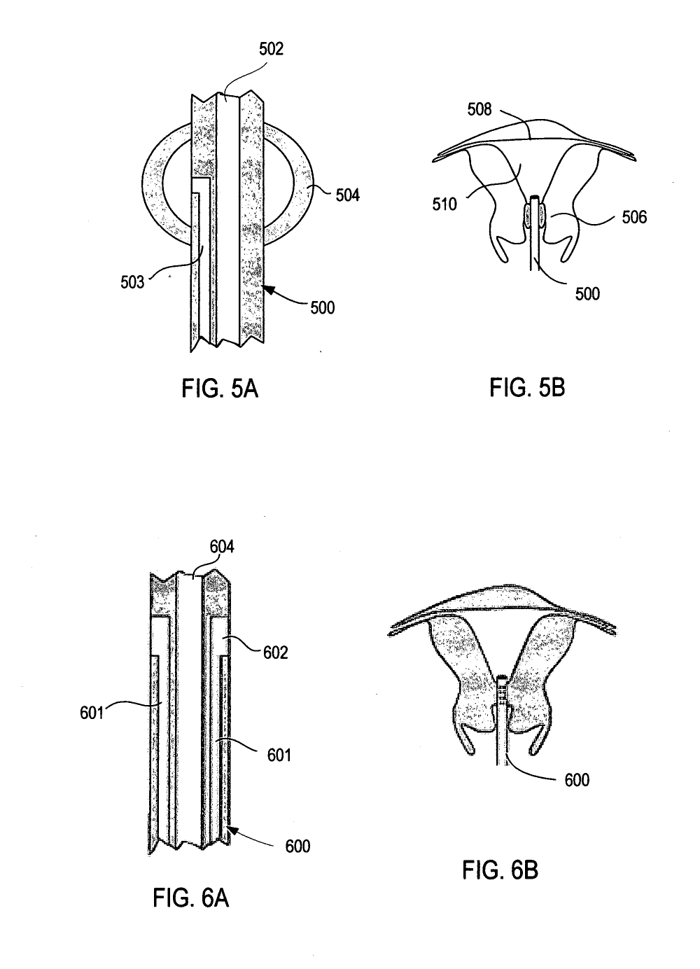

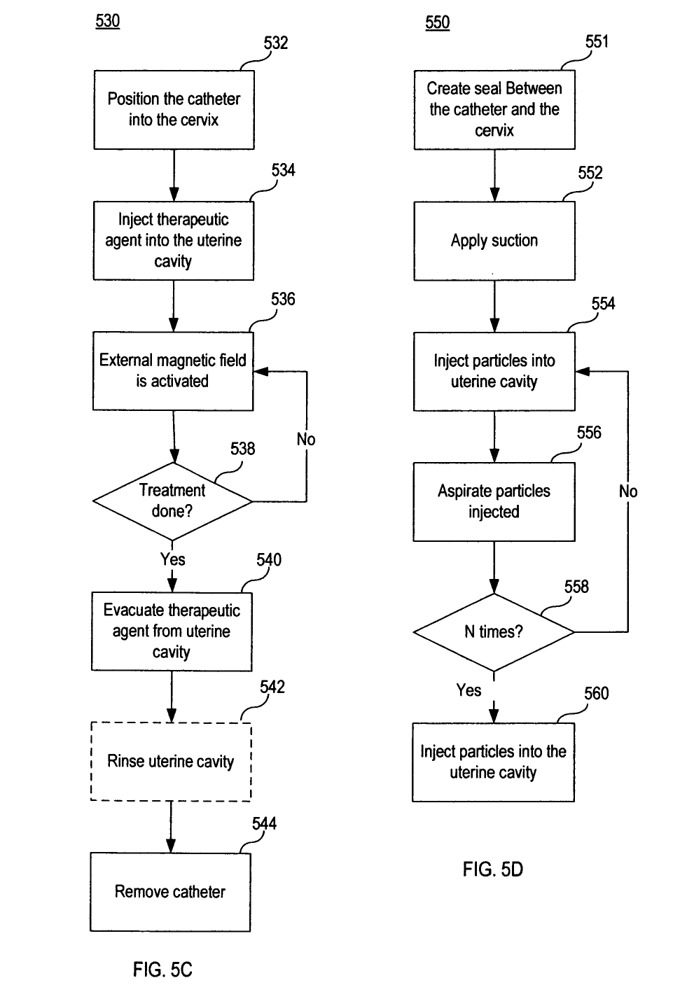

The human body contains numerous body cavities, many of which can be afflicted with diseases that may be effectively treated by applying sufficient thermal energy to destroy or inactivate the malignant cells in the target area. For example, the uterine cavity in a woman's body may develop abnormal uterine bleeding (menorrhagia), which is a common problem for menstruating women. The thermal treatment materials, such as 100, 110, 120, and 140, may be use to perform thermal treatment of the endometrial lining tissue inside the uterine cavity. FIG. 5A shows a schematic cross sectional view of a catheter 500 in accordance with another embodiment of the present invention. FIG. 5B shows a schematic side view of the catheter 500 applied to thermally treat a uterus, more specifically, the endometrial lining tissue 508 of the uterus.

As depicted, the catheter 500 includes lumens 502, 503 and a balloon 504 formed on the outer surface of the catheter. One of the lumens 503 is in fluid communcation with the balloon 504 so that the fluid to inflate the balloon can be introduced via the lumen 503. When inflated, the balloon 504 seals the cervix 506, to therby prevent any leakage of fluid/thermal treatment material outside the uterine cavity 510. The other lumen 502 is used to inject (or evacuate) various types of material into (or from) the uterine cavity 510.

FIG. 5C shows a flow chart 530 illustrating steps to treat the uterus in FIG. 5B in accordance with another embodiment of the present invention. The process starts at a step 532. In the step 532, a physician inserts a delivery system, such as catheter 500, into the uterus. To prevent any leak of the thermal treatment material or fluid outside the uterine cavity 510, the balloon 504 is inflated. The inflated balloon 504 makes a firm contact with the cervix 506 to thereby provide a seal. The device in FIG. 1 of U.S. patent application Ser. No. 11/823,380 can be used to inflate the balloon 504, for instance. Then, in a step 534, the uterine cavity 510 is filled with the thermal treatment material, 100, 110, 120, and 140.

Next, in a step 536, an external alternating electromagnetic field is applied to the thermal treatment material filled in the uterine cavity 510. Then, the particles contained in the thermal treatment material convert the EM energy into thermal energy, where the generated thermal energy is used to ablate the endometrium tissue inside the uterus. An exemplary EM generator disclosed in U.S. patent application Ser. No. 11/823,379, can be used to provide the external EM field. The catheter 500 may be preferably formed of polymer(s) to prevent inadvertent heating.

In a step 538, it is determined whether the thermal treatment is completed. If the answer to the step 538 is NO, the process proceeds to the step 536. Otherwise, the process proceeds to a step 540. In the step 540, the physician removes the thermal treatment material from the uterine cavity 510 by aspiration via the lumen 502. Optionally, the uterine cavity 510 can be flushed with a saline solution in a step 542. Also, any particles remaining in the uterine cavity will be removed within a few days after the treatment via the vagina. Finally, the catheter 500 is removed from the uterus in a step 544.

FIG. 5D shows a flow chart 550 illustrating steps to inject thermal treatment material into the uterine cavity 510 in accordance with another embodiment of the present invention. The process in the flow chart 550 may correspond to the step 534 in FIG. 5C and starts at a step 551. In the step 551, to prevent any leak of the thermal treatment material or fluid outside the uterine cavity 510, the balloon 504 is inflated. Next, in a step 552, a gentle suction is applied through the lumen 502 to aspirate any fluid within the uterine cavity 510 and create a slight vacuum. Then, in a step 554, the uterine cavity 510 is filled with the thermal treatment material. Subsequently, in a step 556, the thermal treatment material filled in the uterine cavity 510 is removed by aspiration via the lumen 502. The steps 554 and 556 form an aspiration/injection cycle.

In a step 558, it is determined whether the aspiration/injection cycle has been repeated a preset number of times. The aspiration/injection cycles correspond to “pressure swings” that ensure the full coverage of thermal treatment material inside the uterine cavity 510. The amount of thermal treatment material delivered into the uterus between successive pressure swings and the pressure inside the delivery system could be used as an indicator of the thermal treatment material coverage. If the answer to the step 558 is NO, the process proceeds to the step 554. Otherwise, the process proceeds to a step 560. In the step 560, the thermal treatment material is injected into the uterine cavity 510.

FIG. 6A shows a schematic cross sectional view of a catheter 600 in accordance with another embodiment of the present invention. FIG. 6B shows a schematic side view of the catheter 600 applied to thermally treat a uterus. As depicted, the catheter 600 is similar to the catheter 500, with the differences that the catheter 600 includes suction holes 602 instead of a balloon and that multiple lumens 601 are connected to the suction holes. When the catheter 600 is inserted into the uterus, the suction holes 602 are positioned in proximity to the cervix. To prevent any leak of thermal treatment material or fluid outside the uterine cavity, a seal can be formed by a vacuum system connected to the suction holes 602 via the lumens 601. The device in FIG. 1 of U.S. patent application Ser. No. 11/823,380 can generate the vacuum, for instance. The low pressure in the lumens 601 causes the inner wall of the cervix to adhere firmly to the catheter 600 around the suction holes 602 thereby to seal the uterine cavity.

The process for treating the uterus in FIG. 6B would be similar to the process in the flow charts 530 and 550, with the difference that, in the step 551, the seal between the cervix and the catheter 600 is formed by use of the suction holes 602 instead of the balloon 504. As such, detailed description of the process for treating the uterus in FIG. 6B is not repeated for brevity.

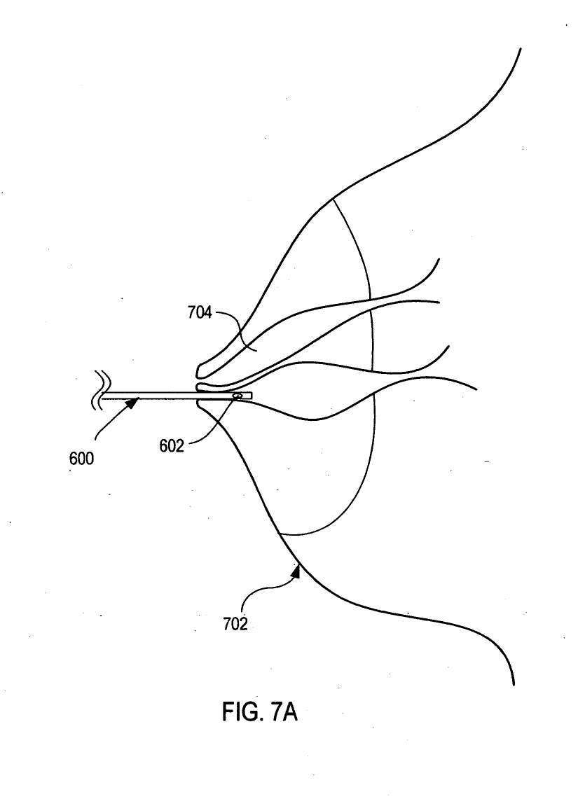

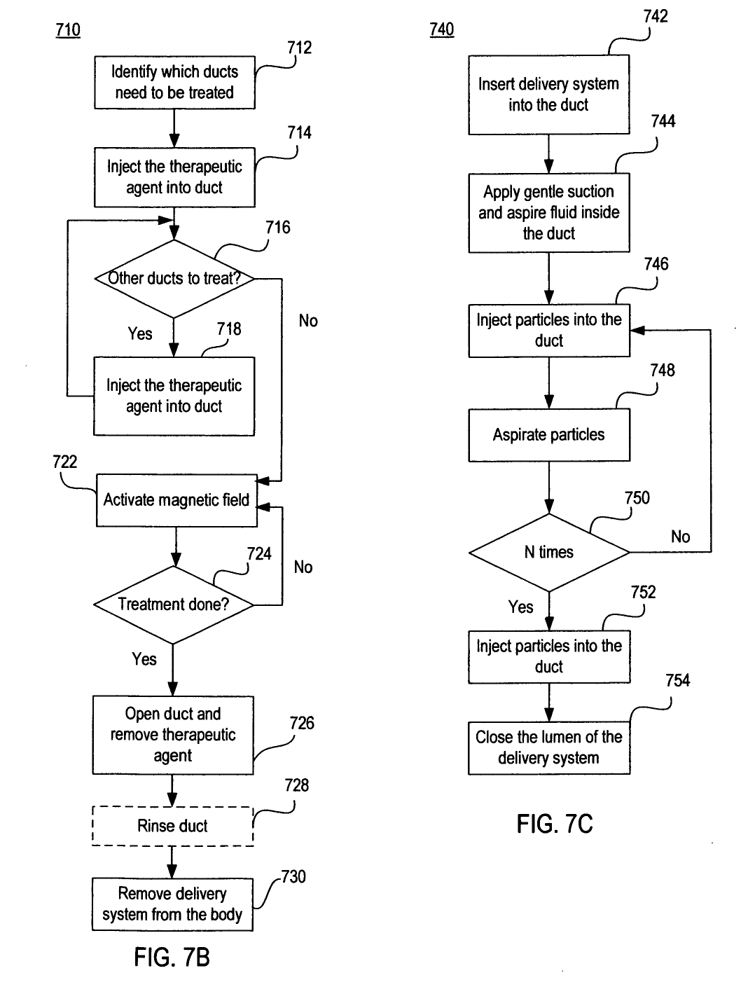

The thermal treatment materials, such as 100, 110, 120, and 140, may be use to perform thermal treatment of the human breast. For instance, intraductal breast cancer (ductal carcinoma in situ—“DCIS”) for patients with atypical ductal hyperplasia (ADH) may be thermally treated by use of the thermal treatment materials. Also, the thermal treatment materials may be used to perform a prophylactic procedure for patients with a high risk of developing breast cancer. FIG. 7A shows a schematic cross sectional view of a human breast 702 with a catheter 600 inserted thereto for thermal treatment in accordance with another embodiment of the present invention.

As depicted in FIG. 7A, the catheter 600 is inserted into the orifice of a milk duct 704 to be thermally treated. Then, a seal between the catheter 600 and the milk duct wall can be formed by a vacuum system connected to the suction holes 602 via the lumens 601, to thereby prevent any leakage of fluid/thermal treatment material outside the milk duct 704. The other lumen 604 may be used to inject (or evacuate) various types of material into (or from) the milk duct 704.

FIG. 7B shows a flow chart 710 illustrating steps to treat the breast 702 in accordance with another embodiment of the present invention. The process starts at a step 712. In the step 712, a physician identifies a milk duct having malignant cells, such as breast cancer, and inserts a thermal treatment material delivery system, such as catheter 600, into the orifice of the identified duct 704. Then, in a step 714, the milk duct 704 is filled with the thermal treatment material, such as 100, 110, 120, and 140. Detailed description of the step 714 is given below in conjunction with FIG. 7C.

Next, in a step 716, a determination is made as to whether or not there is any other milk duct to be treated. If the answer to the step 716 is YES, the process proceeds to a step 718. In the step 718, the thermal treatment material is also injected into the other duct. Then, the process proceeds to the step 716. If the answer to the step 716 is NO, the process proceeds to a step 722.

In the step 722, an external alternating electromagnetic field is applied to the thermal treatment material filled in the milk duct 704. Then, the particles contained in the thermal treatment material convert the EM energy into thermal energy, where the generated thermal energy is transmitted to the surrounding abnormal tissue and destroy it. An exemplary EM generator disclosed in the previously referenced U.S. patent application Ser. No. 11/823,379, can be used to provide the external EM field. The catheter 600 may be preferably formed of polymer(s) to prevent inadvertent heating.

In a step 724, it is determined whether or not the thermal treatment is completed. If the answer to the step 724 is NO, the process proceeds to the step 722. Otherwise, the process proceeds to a step 726. In the step 726, the physician opens the duct, i.e., the duct is injected with pressurized saline so that the duct would expand and allow the particles to be removed from the milk duct 704 by aspiration via the lumen 604. Optionally, the milk duct 704 can be flushed with a saline solution in a step 728. Finally, the catheter 600 is removed from the breast 702 in a step 730.

FIG. 7C shows a flow chart 740 illustrating steps to inject thermal treatment material into the milk duct 704 in accordance with another embodiment of the present invention. The process in the flow chart 740 may correspond to the step 714 in FIG. 7B and starts at a step 742. In the step 742, a delivery system, such as catheter 600, is inserted into the identified milk duct 704. Also, to prevent any leak of the thermal treatment material or fluid outside the milk duct 704, the lumens 601 are evacuated by a vacuum pump connected thereto, causing the inner wall of the milk duct to firmly adhere to the catheter 600. Next, in a step 744, a gentle suction is applied through the lumen 604 to aspirate any fluid within the milk duct 704 and lobules and to create a slight vacuum. Then, in a step 746, the milk duct 704 is filled with the thermal treatment material. Subsequently, in a step 748, the thermal treatment material filled in the milk duct 704 is removed by aspiration via the lumen 604. The steps 746 and 748 form an aspiration/injection cycle.

In a step 750, it is determined whether or not the aspiration/injection cycle has been repeated a preset number of times. The aspiration/injection cycles correspond to “pressure swings” that ensure the full coverage of thermal treatment material inside the milk duct 704. The amount of thermal treatment material delivered into the milk duct between successive pressure swings and the pressure inside the delivery system could be used as an indicator of the thermal treatment material coverage. If the answer to the step 750 is NO, the process proceeds to the step 746. Otherwise, the process proceeds to a step 752. In the step 752, the thermal treatment material is injected into the milk duct 704. Next, in a step 754, the lumen 604 is closed for the subsequent thermal treatment process.

It should be apparent to those of ordinary skill in the art that the size and number of suction holes formed in the catheter 600 may vary according to application. Alternatively, a ring shaped suction hole may be used in place of multiple suction holes. It is noted that the catheter 500 may be used in place of the catheter 600. In such a case, the balloon 504 is used to seal the gap between the catheter and the inner wall of the milk duct.

It is noted that multiple ducts can be treated simultaneously. As an example, a physician could fill two ducts using two different delivery systems, (or, equivalently two catheters), and apply the electromagnetic field around the breast simultaneously. If needed, the physician could select on the EM generator (EM generator is disclosed in the previously referenced U.S. patent application Ser. No. 11/823,379) the number of ducts to treat simultaneously. The EM generator may include information of various types of treatment cycles, each cycle including duration and EM field intensity, etc.

The pressure inside the catheter 500 (or 600) could be monitored to give a feedback to the injection system of the thermal treatment material, where the injection system is disclosed in the previously referenced U.S. patent application Ser. No. 11/823,380. For example, if a pressure outside the working pressure range is detected, the injector will set a warning signal or alarm. For another example, the injector can control the pressure by modulating a valve or pump to maintain an optimum working pressure. Also, if the pressure rises above or falls below a safe limit, the injection system may automatically abort the treatment procedure.

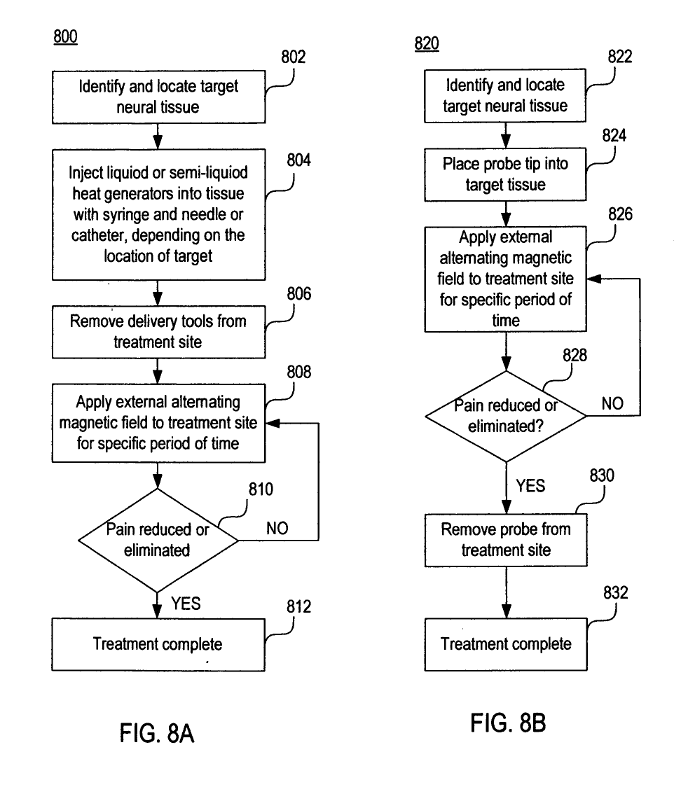

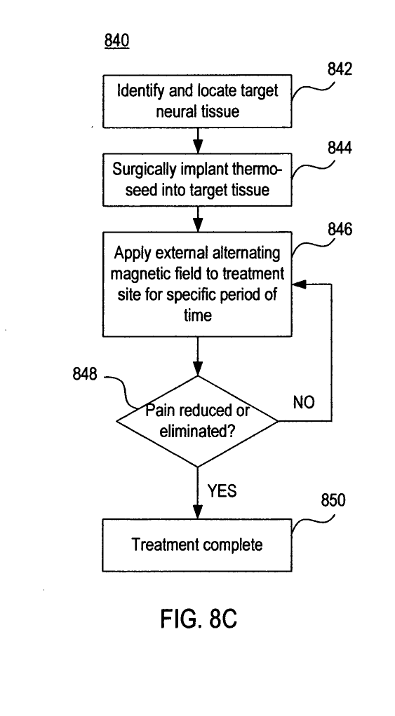

FIG. 8A shows a flow chart 800 illustrating steps to treat neural tissue in accordance with another embodiment of the present invention. The process starts at a step 802. In the step 802, a physician identifies and locates target neural tissue by use of a suitable technique. Then, in a step 804, the thermal treatment material, 100, 110, 120, and 140, is injected into the target neural tissue with a suitable delivery tool, such as syringe, needle, or catheter described in conjunction with FIGS. 2A-7C, depending on the location of the target neural tissue. Next, the delivery tool is removed from the treatment site in a step 806.

In a step 808, an external alternating electromagnetic field is applied to the thermal treatment material. Then, the particles contained in the thermal treatment material convert the EM energy into thermal energy, where the generated thermal energy is transmitted to the target neural tissue and destroy it. An exemplary EM generator disclosed in the previously referenced U.S. patent application Ser. No. 11/823,379, can be used to provide the external EM field. Then, in a step 810, it is determined whether the pain associated with the target neural tissue is reduced or eliminated. If the answer to the step 810 is NO, the process proceeds to the step 808. Otherwise, the process proceeds to a step 812. In the step 812, the treatment is completed.

Neural tissue can be also treated by use of a catheter/probe that includes a heat generator located at its tip. The heat generator is formed of ferrimagnetic, ferromagnetic, or super-paramagnetic material, and secured to the distal end of the catheter. When subject to an alternating EM field applied externally, the heat generator converts the EM energy into thermal energy. Detailed description of catheters having the heat generator can be found in the previously referenced U.S. patent application Ser. No. 11/801,453. FIG. 8B shows a flow chart 820 illustrating steps to treat neural tissue with the catheter having the heat generator in accordance with another embodiment of the present invention. As depicted, the process starts at a step 822.

In the step 822, the target neural tissue is identified and located. Then, in a step 824, the heat generator located at the distal end of the catheter is placed into or in proximity to the target neural tissue. Subsequently, an external alternating EM field is applied to the heat generator for a specific period of time in a step 826. Next, in a step 828, it is determined whether the pain associated with the target neural tissue is reduced or eliminated. If the answer to the step 828 is NO, the process proceeds to the step 826. Otherwise, the process proceeds to a step 830. In the step 830, the catheter is removed from the treatment site. Then, the treatment is completed in a step 832.

FIG. 8C shows a flow chart 840 illustrating steps to treat neural tissue in accordance with another embodiment of the present invention. The process starts at a step 842. In the step 842, the target neural tissue is identified and located. Then, in a step 844, thermo-seed or heat generator is surgically implanted into the target neural tissue, either permanently or short term (bio-absorbable or removed physically). The thermo-seed or heat generator is formed of ferrimagnetic, ferromagnetic, or super-paramagnetic material and generates thermal energy in response to alternating EM field applied thereto externally. Subsequently, an external alternating EM is applied to the thermo-seed or heat generator for a specific period of time in a step 846. Next, in a step 848, it is determined whether or not the pain associated with the target neural tissue is reduced or eliminated. If the answer to the step 848 is NO, the process proceeds to the step 846. Otherwise, the process proceeds to a step 850. In the step 850, the treatment is completed.

In the thermal treatments discussed in conjunction with FIGS. 1A-8C, diseased tissue is treated by elevating the temperature of its individual cells to a lethal level. For instance, temperatures in the range of about 40° C. to about 45° C. can cause irreversible damage to diseased cells, while healthy cells may survive exposure to the temperature up to around 46.5° C. As such, a precise control of the temperature is needed for safe and effective thermal treatments. The thermal treatment materials and heat generators discussed in conjunction with FIGS. 1A-8C are inherently thermally self-regulating to prevent the temperature of the material from exceeding the designed-in upper temperature limit. For instance, the particles contained in the thermal treatment materials and heat generators may be formed of a metal alloy with a preset Curie temperature so that the temperatures of the thermal treatment materials and heat generators do not go beyond the Curie temperature during operation.

The thermal treatment material, such as 100, 110, 120, and 140, can be used to treat fallopian tube occlusion. In another embodiment of the present invention, the thermal energy generated by the thermal treatment material can be used to denature the cellular structure of a portion of the patient's fallopian tubes, to thereby induce collapse and occlusion of the fallopian tubes. This technique prevents the eggs from ovaries from reaching the uterus, creating sterility in females. The technique consists of shrinking the fallopian tube using a category of materials that is capable of converting alternating magnetic field energy into thermal energy. The proteins within the fallopian tube are heated and denatured, resulting in shrinkage of the entire structure. In addition to the heat generating ferrite materials, physical barrier plug materials may be used as an adjunct to further promote the occlusion of the tube.

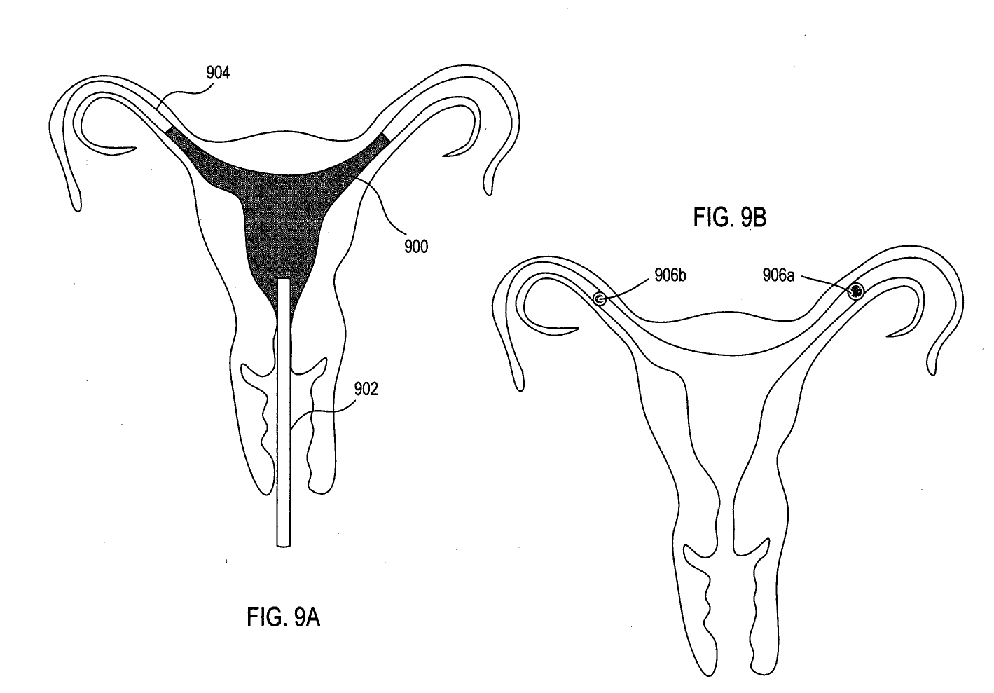

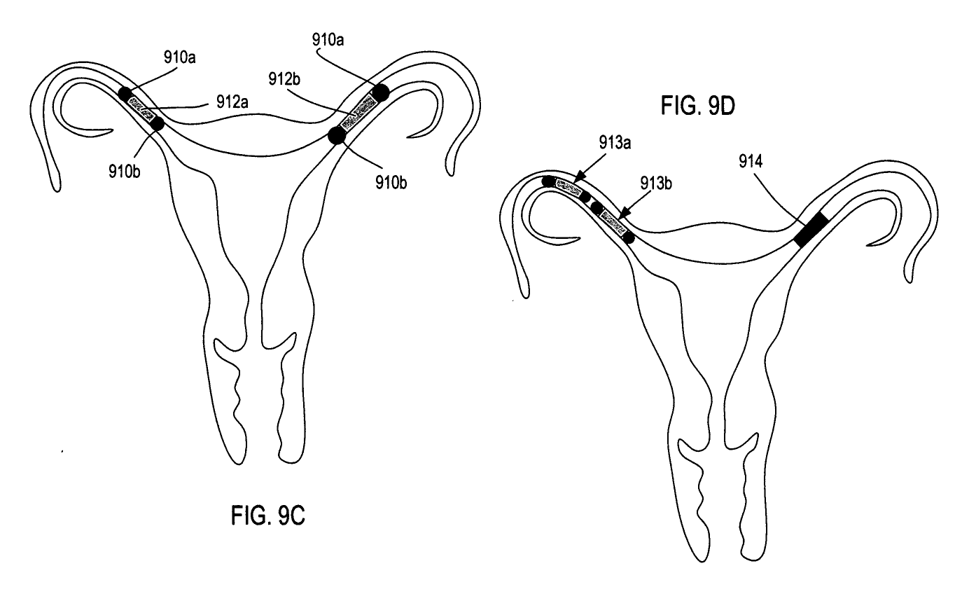

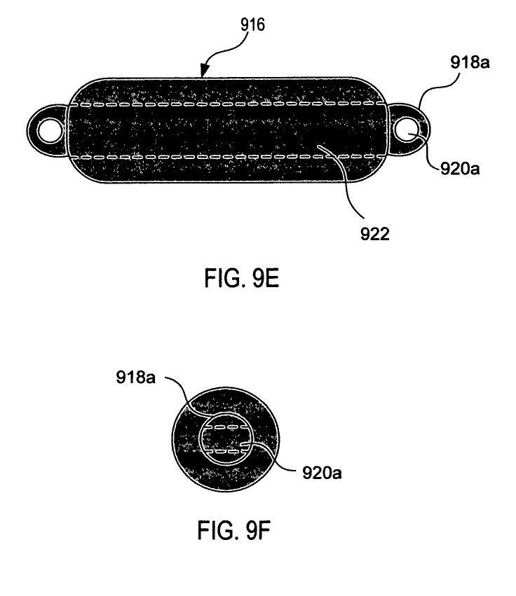

FIG. 9A shows a schematic cross sectional view of a uterus with a catheter 902 inserted thereinto for thermal treatment of fallopian tubes 904 in accordance with another embodiment of the present invention. As depicted, fluid 900, which may include one or more of the thermal treatment materials 100, 110, 120, and 140, is injected via the catheter 902 into the uterine cavity under pressure so that the fluid also partially fills the fallopian tubes 904, where the amount of pressure determines the degree of fluid ingress into the fallopian tube. The catheter 900 may be similar to one of the catheters 500, 506, and 600. Once filled, specific areas are subjected to a local source of alternating magnetic field and subsequently heated. The resulting shrinkage and immune response to the heat induced injury leads to stenosis and occlusion of the lumen.

FIG. 9B shows a schematic cross sectional view of a uterus and fallopian tubes containing a material 906a, 906b for thermal treatment thereof in accordance with another embodiment of the present invention. As depicted, the material 906a, 906b, which may include one or more of the thermal treatment materials 100, 110, 120, and 140, is injected into the midsections of the fallopian tubes using micro catheter. The micro catheter (not shown in FIG. 9C) may be, for instance, a syringe with a long, flexible tube at one end. Then, using an external magnetic field source, the particles in the thermal treatment material 906a, 906b are heated to a predetermined temperature for an appropriate duration. The thermal energy generated by the particles causes the fallopian tube shrink due to the denaturing of collagen and other basement protein. Further, the injury caused by the heating also induces an immune response. The repair mechanism so induced can completely close the lumen rendering the patient infertile.

FIG. 9C shows a schematic cross sectional view of a uterus and fallopian tubes containing materials 910a, 901b and plugs 912a, 912b for thermal treatment thereof in accordance with another embodiment of the present invention. The material 910a, which may include one or more of the thermal treatment materials 100, 110, 120, and 140, is first injected into the fallopian tubes using a specialized micro catheter. Then, plugs 912a, 912b, which is preferably made of bioglass fiber and/or a bioabsorbable material, such as collagen and poly glycolic acid, poly lactic acid, PGLA, etc., is inserted in proximate to the material 910a followed by an injection of second bolus of the material 910b. The material 910b may include one or more of the thermal treatment materials 100, 110, 120, and 140. Alternatively, the materials 910a, 910b and the plug 912a (or 912b) may be formed in one integral body.

The aspect ratio of the plug 912 is determined such that it will always be oriented with its long axis parallel to the fallopian tube. As depicted in FIG. 9C, each plug 912 is sandwiched between two ferrite materials 910a, 910 b. (Hereinafter, the term ferrite material refers to material that can convert EM energy into thermal energy.) After insertion into the fallopian tube, the plugs 912a, 912b are subjected to the alternating magnetic field and the fallopian tubes in contact with the ferrite ends 910a, 901b shrink due to the heat denaturing the proteins and collagen. As the healing process begins, the body will coat the bioglass with epithelial cells there by occluding the lumen. The ferrite materials 901a, 901b at both ends of the plugs 912a, 912b are absorbed and removed from the body via macrophages and eliminated by way of the liver and kidney. This combination of the narrowing and further immune response mediate healing compromises the patency of the fallopian tube. The plugs 912a, 912b act as added barrier to the passage of egg to the uterus. Further, the narrowing of the fallopian tube on both sides of each plug prevents the movement of the plug.

FIG. 9D shows a schematic cross sectional view of a uterus and fallopian tubes containing plug units 913a, 913b and a plug 914 for thermal treatment thereof in accordance with another embodiment of the present invention. Each of the plug units 913a, 913b includes a plug interposed, between two ferrite materials. Shrinkage of the fallopian tube by inductive heating of the two plug units 913a, 913b creates a multiple barrier, decreasing the probability of the eggs reaching the uterus. Also, as depicted in FIG. 9D, only a plug 914, which id formed of ferrite material, is inserted into the fallopian tube. Then, an external alternating EM field is applied so that the plug 914 generates thermal energy, inducing the shrinkage of the fallopian tube. In this approach, the plug 914 acts, as heating element as well as the physical barrier.

FIG. 9E shows a schematic side view of a plug 916 for thermal treatment of a fallopian tube in accordance with another embodiment of the present invention. FIG. 9F shows a schematic end view of the plug 916. The plug 916 is introduced into the fallopian tube and heated inductively from an external alternating EM source. Heating is controlled such away that there is enough shrinkage of the fallopian tube to hold the plug 916 without completely closing the lumen. The plug 916 creates the physical barrier for passage of matured egg to the uterus. When reversal of the sterilization is desired, the inner core 922 of plug is removed by a suitable grasping device, such as a needle holder with luer-lock cleaning port, reopening a clear passage for the egg. This is achieved by again mildly heating, the plug with an external alternating EM source while physically pulling the inner core with the grasping device. The plug 916 includes a handle 918a with an opening 920a, which aids in grasping the inner core 922 of the plug 916. The grasping device may be formed of material that does not respond to the external EM field.

It is noted that the external EM field applied to the ferrite materials and plugs in FIGS. 9A-9F can be generated by a device described in the previously referenced U.S. patent application Ser. No. 11/823,379. As such, the description of the EM field generation device is not repeated for brevity.

It should be understood, of course, that the foregoing relates to exemplary embodiments of the invention and that modifications may be made without departing from the spirit and scope of the invention as set forth in the following claims.

What is claimed is:

1. A material to be injected into a target tissue of a body, comprising:
a carrier substrate;
a plurality of first particles operative to generate thermal energy in response to an alternating electromagnetic field applied external to the body; and
a plurality of second particles, each said second particle having a core and a coating surrounding said core and to be dissolved at a preset temperature by the thermal energy, visibility of said core in an external imaging system being affected as said coating is dissolved to expose said core,
whereby a variation of the visibility can be used as an indicator to determine if the material has reached the preset temperature.

2. A material as recited in claim 1, wherein the visibility of said core increases as the coating is dissolved.

3. A material as recited in claim 1, wherein the visibility of said core decreases as the coating is dissolved.

4. A material as recited in claim 1, wherein the external imaging system is selected from the group consisting of ultrasound, fluoroscopy, and MRI.

5. A material as recited in claim 1, wherein the core is formed of a material selected from the group of Gadolinium based material, Methylxanthines, an N-acetylcysteine.

6. A material as recited in claim 1, further comprising:
a plurality of third particles, each said third particle including a core formed of therapeutic agent and a therapeutic agent coating surrounding the therapeutic agent, the therapeutic agent coating being adapted to release or activate the therapeutic agent when the therapeutic agent coating is heated.

7. A material as recited in claim 1, wherein the carrier substrate is formulated as a liquid, gel, solid, or a permutation thereof,

8. A material as recited in claim 1, wherein the carrier substrate is formed of bio-absorbable material.

9. A material as recited in claim 1, wherein a viscosity of the carrier substrate increases when a static external magnetic field is applied thereto.

10. A material as recited in claim 9, wherein the carrier substrate becomes a viscoelastic solid when the static external magnetic field is applied thereto.

11. A material as recited in claim 1, wherein the plurality of first particles are formed of a material selected from the group of ferrimagnetic, ferromagnetic, and super-paramagnetic materials.

12. A material as recited in claim 1, wherein the plurality of first particles are formed of a material having a preset Curie temperature and wherein the preset Curie temperature is lower than a threshold temperature to damage healthy cells and higher than a threshold temperature to destroy malignant cells.

13. A material as recited in claim 1, wherein an average size of the plurality of the first particles ranges from 1 nm to 100 μm.

14. A material as recited in claim 1, wherein one or more of the plurality of the first particles are surrounding by a surfactant coating.
